(19) United States
(12) Patent Application Publication
(10) Pub. No.: US 2008/0206169 A1
(43) Pub. Date: Aug. 28, 2008

(54) Personal care composition

(76) Inventors: Cheri Lynn Millikin, West Chester, OH (US); Donald Lynn Bissett, Hamilton, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/985,383

(22) Filed: Nov. 15, 2007

Related U.S. Application Data
(60) Provisional application No. 60/859,361, filed on Nov. 16, 2006.

(51) Int. Cl. A61K 8/30; A61K 31/21; A61K 31/352; A61K 31/22; A61K 31/44; A61K 31/7028; A61K 31/045; A61K 31/52; A61Q 19/00; A61Q 17/04; A61K 31/195; A61K 36/82; A61K 31/155; A61K 31/355; A61K 36/06; A61K 38/02; A61K 31/70

(52) U.S. Cl. 424/59; 514/506; 514/460; 514/547; 514/2; 514/23; 424/195.16; 514/357; 514/458; 514/27; 514/636; 514/731; 514/729; 424/729; 514/263.34; 514/567; 514/563; 514/33

(57) ABSTRACT

Personal care composition comprising at least one skin care active selected from the group consisting of acetyl glutamic acid, acetyl glutamine, acetyl methionine, acetyl tributyl citrate, acetyl triethyl citrate, acetyl tyrosine, adipic acid, alanine, arginine, arginine glutamate, benzophenone-3, camphor, gluconolactone, glucose, glycine, histidine hydrochloride, hydroxyproline, maltitol, phenylalanine, succinic acid, buffered lactic acid, tris(tetramethylhydroxypiperidinol) citrate, a boswellic acid compound, and salts, isomers, derivatives, and mixtures of any of the foregoing; and a dermatologically acceptable carrier.

### CROSS REFERENCE TO RELATED APPLICATION

This application claims the benefit of U.S. provisional application Ser. No. 60/859,361 filed on Nov. 16, 2006, which is herein incorporated by reference.

### FIELD OF THE INVENTION

The present invention relates to personal care compositions comprising at least one skin care active useful for regulating the condition of mammalian keratinous tissue. The present invention further relates to methods of use of the personal care composition, a kit, an article of commerce, and a method of marketing the personal care composition described herein.

### BACKGROUND OF THE INVENTION

A number of personal care products, which are directed toward improving the health and physical appearance of keratinous tissues such as the skin, hair, and nails, currently are available to consumers. Many of these products meet the continuing need of regulating the condition of keratinous tissue by delaying, minimizing or even eliminating skin wrinkling and histological changes typically associated with the aging of skin or environmental damage to human skin. In addition, there exists a need for cosmetic agents to prevent, retard, and/or treat uneven skin tone by acting as a skin lightening or pigmentation reduction cosmetic agent.

### SUMMARY OF THE INVENTION

The present invention meets the aforementioned needs. Applicants believe that personal care compositions comprising skin care actives described herein may be used to provide prophylactic as well as therapeutic regulation of keratinous tissue conditions. The compositions of the present invention may be particularly useful for skin-lightening.

The following describe some non-limiting embodiments of the present invention.

According to a first embodiment of the present invention, a personal care composition is provided comprising at least one skin care active selected from the group consisting of acetyl triethyl citrate, gluconolactone, and mixtures thereof; a dermatologically acceptable carrier; and at least one additional skin care active selected from the group consisting of vitamin B compounds, vitamin E compounds, vitamin C compounds, vitamin D compounds, peptides, sugar amines, protease inhibitors, sunscreens, desquamation agents, chelators, skin lightening compounds, non-vitamin antioxidant radical scavengers, phytosterols, hesperidin compounds, retinoid compounds, plant hormones, protease inhibitors, tyrosinase inhibitors, anti-inflammatory agents, topical anesthetics, anti-cellulite agents, sunless tanning agents, N-acyl amino acid compounds, yeast extract, and salts, isomers, derivatives and mixtures of any of the foregoing.

According to a second embodiment of the present invention, a method of regulating the condition of mammalian keratinous tissue is provided comprising the step of topically applying to the keratinous tissue of a mammal needing such treatment a personal care composition according to the first embodiment. In one embodiment the method comprises the step of delivering energy to the keratinous tissue. In an alternative embodiment, the composition is selectively applied to keratinous tissue exhibiting increased pigmentation relative to the surrounding tissue.

According to yet another embodiment of the present invention, a kit is provided comprising a composition according to the first embodiment, and at least one additional component.

According to yet another embodiment of the present invention, an article of commerce is provided comprising a personal care composition according to the first embodiment; packaging for the personal care composition and/or advertisement material pertaining to the personal care composition. The advertisement material may comprise images comparing the appearance of a person prior to use of the composition to the appearance of the same person after use of the composition. Additionally or alternatively, the advertisement material may comprise indicia and/or an image which communicates that the personal care composition can be used in conjunction with an energy delivery device for regulating the condition of mammalian keratinous tissue.

According to yet another embodiment of the present invention, a method of marketing a personal care composition according to the first embodiment for preventing, retarding, and/or treating uneven skin tone is provided. In one embodiment, the method of marketing comprises the step of making available to a consumer the personal care composition, and providing a communication to the consumer that the topical application of the personal care composition may improve the consumer's skin tone.

### DETAILED DESCRIPTION OF THE INVENTION

The compositions of the present invention may be used in a variety of personal care products, non-limiting examples of which include moisturizers, conditioners, cleansers, sunscreens, anti-aging compounds, cosmetics (including lipstick, foundation, rouges, and/or mascara), and combinations thereof. The composition may be in a variety of forms, including but not limited to an emulsion, lotion, milk, liquid, solid, cream, gel, mousse, ointment, paste, serum, stick, spray, tonic, aerosol, foam, pencil, etc. The compositions of the present invention also may be in the form of shave prep products, including, for example, gels, foams, lotions, and creams; and include both aerosols and non-aerosols versions. In one embodiment, the composition is in a form suitable for use with an implement, for example, such as a swab and/or a pen.

In all embodiments of the present invention, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. All measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

“Personal care composition,” as used herein, means compositions suitable for topical application on mammalian keratinous tissue.

“Skin care actives,” or “actives,” as used herein, means compounds that, when applied to the skin, provide a benefit or improvement to the skin. It is to be understood that skin care actives are useful not only for application to skin, but also to hair, nails and other mammalian keratinous tissue.

“Keratinous tissue,” as used herein, refers to keratin-containing layers disposed as the outermost protective covering of mammals which includes, but is not limited to, skin, hair, nails, cuticles, etc.

“Dermatologically acceptable,” as used herein, means that the compositions or components described are suitable for use in contact with human keratinous tissue without undue toxicity, incompatibility, instability, allergic response, and the like.

“Effective amount” as used herein means an amount of a compound or composition sufficient to significantly induce a positive benefit, preferably a positive keratinous tissue appearance or feel benefit, including independently or in combination the benefits disclosed herein, but low enough to avoid serious side effects (i.e., to provide a reasonable benefit to risk ratio, within the scope of sound judgment of the skilled artisan).

“Delivery enhancement device,” as used herein, means any device that increases the amount of active ingredient applied to and/or into the skin relative to the amount of active ingredient that is delivered without using the device.

“Regulating skin condition,” as used herein, means improving skin appearance and/or feel, for example, by providing a smoother appearance and/or feel. Herein, “improving skin condition” means effecting a visually and/or tactilely perceptible positive change, or benefit, in skin appearance and feel. Without wishing to be limited by theory, typical skin damage includes thinning of the skin, which occurs naturally as one ages. With such thinning, there is a reduction in the cells and blood vessels that supply the skin as well as a flattening of the dermal-epidermal junction that results in weaker mechanical resistance of this junction. See, for example, Oikarinen, “The Aging of Skin: Chronoaging Versus Photoaging,” Photodermatol. Photoimmunol. Photomed., vol. 7, pp. 3-4, 1990. Benefits that may be provided include, but are not limited to, one or more of the following: Reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); skin lightening; preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by hyperpigmentation, etc. As used herein, “regulating skin condition” is intended to include regulation of such signs irrespective of the mechanism of origin. For example, skin lightening may occur through multiple mechanisms including anti-oxidant mechanisms, trypsin inhibition, anti-inflammatory mechanisms, nitric oxide scavenging, tyrosinase inhibition, etc.

“Signs of skin aging,” as used herein, include but are not limited to, all outward visibly and tactilely perceptible manifestations, as well as any macro- or microeffects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; loss of skin elasticity; discoloration (including undereye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

“Hyperpigmentation,” as used herein, refers to an area of skin wherein the pigmentation is greater than that of an adjacent area of skin (e.g., a pigment spot, an age spot, and the like).

“Desquamation, exfoliation, and/or turnover,” as used herein, mean the removal of the upper layers of the stratum corneum (comprising the horny layers).

“Oily and/or shiny appearance,” as used herein, mean the glossy look mammalian skin tends to exhibit upon the excretion of oil, sebum, and/or sweat from the respective source gland.

“Sagging,” as used herein, means the laxity, slackness, or the like condition of skin that occurs as a result of loss of, damage to, alterations to, and/or abnormalities in dermal elastin.

“Smoothing” and softening,” as used herein, means altering the surface of the keratinous tissue such that its tactile feel is improved.

“Sallowness,” as used herein means the pale color, yellow color or the like condition of skin that occurs as a result of a loss of, damage to, alterations to, and/or abnormalities in skin components such that they become colored (e.g., yellow in color) due to processes such as protein glycation and accumulation of lipofuscin or in the decrease in peripheral blood flow that typically accompanies skin aging.

“Derivatives,” as used herein, includes but is not limited to, amide, ether, ester, amino, carboxyl, acetyl, and/or alcohol derivatives of a given compound.

“Isomers,” as used herein, is understood to include D-isomers, L-isomers, and/or DL-isomers of a given compound.

“Salts,” as used herein, includes but is not limited to sodium, potassium, calcium, ammonium, manganese, copper, and/or magnesium salts of a given compound.

### I. Personal Care Composition

### Skin Care Actives

The composition of the present invention comprises at least one skin care active selected from the group consisting of acetyl glutamic acid, acetyl glutamine, acetyl methionine, acetyl tributyl citrate, acetyl triethyl citrate, acetyl tyrosine, adipic acid, alanine, arginine, arginine glutamate, benzophenone-3, camphor, gluconolactone, glucose, glycine, histidine hydrochloride (HCl), hydroxyproline, maltitol, phenylalanine, succinic acid, buffered lactic acid, tris(tetramethylhydroxypiperidinol) citrate, a boswellic acid compound, their salts, isomers, derivatives, and mixtures thereof. In one embodiment, the isomer is an L-isomer.

The composition of the present invention may comprise from about 0.01% to about 10%, alternatively from about 0.1% to about 7%, alternatively from about 0.1% to about 5%, and alternatively from about 0.5% to about 3%, of one or more of the following compounds, their salts, isomers, and/or derivatives, all commercially available from Sigma Aldrich, St. Louis, Mo.: Acetyl glutamic acid, CAS 5817-08-3, useful for increasing the intracellular content of glutathione, an anti-oxidant, an inhibitor of tyrosinase, and a nitric oxide scavenger; acetyl aglutamine, CAS 2490-97-3, useful for increasing the intracellular content of glutathione, an anti-oxidant, an inhibitor of tyrosinase, and a nitric oxide scavenger; acetyl methionine, CAS 1115-47-5, useful as an anti-oxidant, for increasing the intracellular content of glutathione, for activating glutathione reductase, and as a nitric oxide scavenger; acetyl tributyl citrate, or tributyl 2-acetylcitrate, CAS 77-90-7, useful for increasing the intracellular content of glutathione and for activating glutathione reductase; acetyl triethyl citrate, or triethyl 2-acetylcitrate, CAS 77-89-4, useful for increasing the intracellular content of glutathione and for activating glutathione reductase; acetyl tyrosine, CAS 537-55-3, useful as an anti-oxidant, an inhibitor of tyrosinase, and a nitric oxide scavenger; adipic acid, CAS 124-04-9, useful for increasing the intracellular content of glutathione, as an anti-oxidant, an inhibitor of tyrsoinase, and a nitric oxide scavenger; alanine, CAS 338-69-2, useful for increasing the intracellular content of glutathione as well as an activating glutathione reductase; arginine glutamate, CAS 4320-30-3, useful for increasing the intracellular content of glutathione, for activating glutathione reductase, and as an inhibitor of tyrosinase; benzophenone-3, CAS 131-57-7, useful for increasing the intracellular content of glutathione, as an anti-oxidant with anti-inflammatory properties, an inhibitor of COX 2, an inhibitor of tyrosinase, and an inhibitor of trypsin; camphor, CAS 76-22-2, useful for increasing the intracellular content of glutathione, for activating glutathione reductase, and as an inhibitor of tyrosinase; gluconolactone, CAS 90-80-2, useful for increasing the intracellular content of glutathione, activating glutathione reductase, and as an anti-oxidant and a nitric oxide scavenger; glucose, CAS 50-99-7, useful for increasing the intracellular content of glutathione and for activating glutathione reductase; glycine, CAS 56-40-6, useful for increasing the intracellular content of glutathione, activating glutathione reductase, and as an inhibitor of COX 2; histidine HCl, CAS 351-50-08, useful for increasing the intracellular content of glutathione, activating glutathione reductase, and as an inhibitor of COX 2; hydroxyproline CAS 51-35-4, useful for increasing the intracellular content of glutathione, activating glutathione reductase, as an anti-oxidant with anti-inflammatory properties, an inhibitor of COX 2, and a nitric oxide scavenger; maltitol, CAS 585-88-6, useful for increasing the intracellular content of glutathione, activating glutathione reductase as an inhibitor of tyrosinase; phenylalanine, CAS 63-91-2, useful for increasing the intracellular content of glutathione, activating glutathione reductase, as an anti-oxidant with anti-inflammatory properties, an inhibitor of COX 2, and a nitric oxide scavenger; succinic acid, CAS 110-15-6, useful for increasing the intracellular content of glutathione, activating glutathione reductase, as an anti-oxidant, an inhibitor of tyrosinase, and a nitric oxide scavenger; and arginine, CAS 157-06-2, useful for increasing the intracellular content of glutathione, activating glutathione reductase and as an anti-oxidant and nitric oxide scavenger.

Additionally or alternatively, the composition of the present invention may comprise from about 0.01% to about 10%, alternatively from about 0.1% to about 7%, alternatively from about 0.1% to about 5%, and alternatively from about 0.5% to about 3%, of a boswellic acid compound, useful as an ant-inflammatory and analgesic. One example of a suitable boswellic acid-containing compound is Boswellin™ (Sabinsa Corp., Piscataway, N.J.).

Additionally or alternatively, the composition of the present invention may comprise from about 0.01% to about 10%, alternatively from about 0.1% to about 7%, alternatively from about 0.1% to about 5%, and alternatively from about 0.5% to about 3%, of lactic acid, its salts, isomers, and/or derivatives. The lactic acid may be buffered at a pH of from about 5 to about 7 (referred to herein as “buffered lactic acid”), and is useful as an activator of glutathione reductase, an anti-oxidant, an inhibitor of COX I and COX 2, an inhibitor of tyrosinase, an inhibitor of trypsin, and a nitric oxide scavenger. One example of a commercially available buffered lactic acid is Purac™ BF/P 41, available from Purac, Lincolnshire, Ill.

Additionally or alternatively, the composition of the present invention may comprise from about 0.01% to about 10%, alternatively from about 0.1% to about 7%, alternatively from about 0.1% to about 5%, and alternatively from about 0.5% to about 3%, of tris(tetramethylhydroxypiperidinol) citrate, its salts, isomers, and/or derivatives, useful as an anti-oxidant and an inhibitor of COX 2 and COX 1. One example of commercially available tris(tetramethylhydroxypiperidinol) citrate is Tinogard Q™, available from Ciba Specialty Chemicals, Terrytown, N.Y.

Additionally or alternatively, the composition of the present invention may comprise from about 0.01% to about 10%, alternatively from about 0.1% to about 7%, alternatively from about 0.1% to about 5%, and alternatively from about 0.5% to about 3%, of gluconolactone, and/or its derivatives, useful as a skin lightener, and/or reducing skin redness and/or yellowness. One commercially available gluconolactone is ADM GDL™, available from Archer Daniels Midland Company, Decatur, Ill.

Additionally or alternatively, the composition of the present invention may comprise from about 0.01% to about 10%, alternatively from about 0.1% to about 7%, alternatively from about 0.1% to about 5%, and alternatively from about 0.5% to about 3%, of acetyl triethyl citrate, and/or its derivatives, useful as a skin lightener, and/or reducing skin redness and/or yellowness. One commercially available acetyl triethyl citrate is Citroflex A-2™, available from Morflex, Inc., Greensboro, N.C.

### Additional Skin Care Actives

The compositions of the present invention comprise at least one additional skin care active, useful for regulating and/or improving the condition of mammalian skin. Classes of suitable skin care actives include, but are not limited to vitamins, peptides and peptide derivatives, sugar amines, sunscreens, oil control agents, particulates, flavonoid compounds, hair growth regulators, antioxidants and/or anti-oxidant precursors, preservatives, phytosterols, protease inhibitors, tyrosinase inhibitors, anti-inflammatory agents, and mixtures thereof. It should be noted, however, that many skin care actives may provide more than one benefit, or operate via more than one mode of action. Therefore, classifications herein are made for the sake of convenience and are not intended to limit the active to that particular application or applications listed.

### A. Vitamins

The compositions of the present invention may comprise from about 0.0001% to about 50%, alternatively from about 0.001% to about 10%, alternatively from about 0.01% to about 5%, and alternatively from about 0.1% to about 1%, of one or more vitamins. Herein, “vitamins” means vitamins, pro-vitamins, and their salts, isomers and derivatives. Non-limiting examples of suitable vitamins include: vitamin B compounds (including B1 compounds, B2 compounds, B3 compounds such as niacinamide, niacinnicotinic acid, tocopheryl nicotinate, C1-C18 nicotinic acid esters, and nicotinyl alcohol; B5 compounds, such as panthenol or “pro-B5”, pantothenic acid, pantothenyl; B6 compounds, such as pyroxidine, pyridoxal, pyridoxamine; carnitine, thiamine, riboflavin); vitamin A compounds, and all natural and/or synthetic analogs of Vitamin A, including retinoid compounds such as retinol, retinyl acetate, retinyl palmitate, retinoic acid, retinaldehyde, retinyl propionate; carotenoids (pro-vitamin A); vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids. In one embodiment, the composition comprises a vitamin selected from the group consisting of vitamin B compounds, vitamin C compounds, vitamin E compounds and mixtures thereof. Alternatively, the vitamin is selected from the group consisting of niacinamide, tocopheryl nicotinate, pyroxidine, panthenol, vitamin E, vitamin E acetate, ascorbyl phosphates, ascorbyl glucoside, and mixtures thereof.

### B. Peptides and Peptide Derivatives

The compositions of the present invention may comprise one or more peptides. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins, carnosine (beta-alanine-histidine), palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, France, acetyl-glutamate-glutamate-methionine-glutamine-arginine-arginine (Ac-EEMQRR; Argireline®), and Cu-histidine-glycine-glycine (Cu-HGG, also known as IAMIN®).

The compositions may comprise from about 1×10⁻⁷% to about 20%, alternatively from about 1×10⁻⁶% to about 10%, and alternatively from about 1×10⁻⁵% to about 5% of the peptide.

### C. Sugar Amines

The compositions of the present invention may comprise a sugar amine, also known as amino sugars, and their salts, isomers, tautomers and derivatives. Sugar amines can be synthetic or natural in origin and can be used as pure compounds or as mixtures of compounds (e.g., extracts from natural sources or mixtures of synthetic materials). For example, glucosamine is generally found in many shellfish and can also be derived from fungal sources. Sugar amine compounds useful in the present invention include, for example, N-acetyl-glucosamine, and also those described in PCT Publication WO 02/076423 and U.S. Pat. No. 6,159,485, issued to Yu, et al. In one embodiment, the composition comprises from about 0.01% to about 15%, alternatively from about 0.1% to about 10%, and alternatively from about 0.5% to about 5%, of the sugar amine.

### D. Sunscreens

The compositions of the subject invention may comprise one or more sunscreen actives (or sunscreen agents) and/or ultraviolet light absorbers. Herein, “sunscreen active” includes both sunscreen agents and physical sunblocks. Sunscreen actives and ultraviolet light absorbers may be organic or inorganic. Examples of suitable sunscreen actives and ultraviolet light absorbers are disclosed in The Cosmetic, Toiletry, and Fragrance Association's The International Cosmetic Ingredient Dictionary and Handbook, 10^{th} Ed., Gottschalck, T.E. and McEwen, Jr., Eds. (2004), p. 2267 and pp. 2292-93. Particularly suitable sunscreen actives include benzophenone, benzophenone-1, benzophenone-2, benzophenone-3, benzophenone-4, benzophenone-5, benzophenone-6, benzophenone-7, benzophenone-8, benzophenone-9, benzophenone-10, benzophenone-11, benzophenone-12, benzotriazolyl dodecyl p-cresol, 3-benzylidene camphor, benzylidene camphor sulfonic acid, benzyl salicylate, bis-ethylhexyloxyphenol methoxyphenyl triazine, bornelone, bumetrizole, butyl methoxydibenzoyl-methane, butyl PABA (p-aminobenzoic acid), cinnamidopropyl-trimonium chloride, cinoxate, dea-methoxycinnamate, dibenzoxazoyl naphthalene, di-t-butyl hydroxy-benzylidene camphor, diethylamino hydroxy-benzoyl hexyl benzoate, diethylhexyl butamido triazone, diethylhexyl 2,6-naphthalate, diisopropyl ethyl cinnamate, diisopropyl methyl cinnamate, di-methoxycinnamido-propyl ethyldimonium chloride ether, dimethyl PABA ethyl cetearyldimonium tosylate, dimorpholino-pyridazinone, dimorpholino-pyridazinone, disodium bisethylphenyl triaminotriazine stilbenedisulfonate, disodium distyrylbiphenyl disulfonate, disodium phenyl dibenzimidazole tetrasulfonate, drometrizole, drometrizole trisiloxane, ethyl dihydroxypropyl PABA, ethyl diisopropyl-cinnamate, ethylhexyl bis-isopentylbenzoxazolylphenyl melamine, ethyl dimethoxybenz-ylidene dioxoimidazolidine propionate, ethylhexyl dimethyl PABA, ethylhexyl methoxy-cinnamate, ethylhexyl methoxydibenzoyl-methane, ethylhexyl salicylate, ethylhexyl triazone, ethyl methoxycinnamate, ethyl PABA, ethyl urocanate, etocrylene, 4-(2-beta-glucopyrano-siloxy) propoxy-2-hydroxybenzophenone, glyceryl ethylhexanoate dimethoxycinnamate, glyceryl PABA, glycol salicylate, hexanediol disalicylate, homosalate, isoamyl cinnamate, isoamyl p-methoxycinnamate, isopentyl trimethoxy-cinnamate trisiloxane, isopropylbenzyl salicylate, isopropyl dibenzoylmethane, isopropyl methoxy-cinnamate, kaempferia galanga root extract, menthyl anthranilate, menthyl salicylate, methoxycinnamido-propyl hydroxysultaine, methoxycinnamido-propyl laurdimonium tosylate, 4-methylbenzylidene camphor, methylene bis-benzotriazolyl tetramethylbutyl-phenol, octocrylene, octrizole, PABA, PEG-25 PABA, phenylbenzimidazole sulfonic acid, polyacrylamidomethyl benzylidene camphor, polyamide-2, polyquaternium-59, polysilicone-15, potassium methoxy-cinnamate, potassium phenyl-benzimidazole sulfonate, red petrolatum, sodium benzotriazoyl butylphenol sulfonate, sodium phenylbenz-imidazole sulfonate, sodium urocanate, TEA-phenylbenzimidazole sulfonate, TEA-salicylate, terephthalylidene dicamphor sulfonic acid, tetrabutyl phenyl hydroxybenzoate, titanium dioxide, urocanic acid, zinc cerium oxide, zinc oxide, and mixtures thereof. In one embodiment, the composition comprises from about 1% to about 20%, and alternatively from about 2% to about 10% by weight of the composition, of the sunscreen active and/of ultraviolet light absorber. Exact amounts will vary depending upon the chosen sunscreen active and/or ultraviolet light absorber and the desired Sun Protection Factor (SPF), and are within the knowledge and judgment of one of skill in the art.

### E. Oil Control Agents

The compositions of the present invention may comprise one or more compounds useful for regulating the production of skin oil, or sebum, and for improving the appearance of oily skin. Examples of suitable oil control agents include salicylic acid, dehydroacetic acid, benzoyl peroxide, vitamin B3 compounds (for example, niacinamide or tocopheryl nicotinate), their isomers, esters, salts and derivatives, and mixtures thereof. The compositions may comprise from about 0.0001% to about 15%, alternatively from about 0.01% to about 10%, alternatively from about 0.1% to about 5%, and alternatively from about 0.2% to about 2%, of an oil control agent.

### F. Particulates

The compositions of the present invention may comprise one or more particulate materials. Nonlimiting examples of particulate materials useful in the present invention include colored and uncolored pigments, interference pigments (nonlimiting examples include mica, layered with about 50-300 nm films of TiO₂, Fe₂O₃ silica, tin oxide, Cr₂O₃, and mixtures thereof, spherical TiO₂ particles having a size of from about 100 to about 300 nanometers; or alternatively, spherical TiO₂ particles having a size of from about 1 to about 30 micrometers; and mixtures thereof), inorganic powders (for example, iron oxides, zinc oxides, ferric ammonium ferrocyanide, manganese violet, ultramarine blue, and chrome oxide), organic powders (for example, phthalocyanine blue and green pigment), composite powders, optical brightener particles, and combinations thereof. These particulates can, for instance, be platelet shaped, spherical, elongated or needle-shaped, or irregularly shaped; surface coated or uncoated; porous or non-porous; charged or uncharged; and can be added to the current compositions as a powder or as a pre-dispersion.

In one embodiment, the compositions may comprise from about 0.01% to about 20%, alternatively from about 0.05% to about 10%, alternatively from about 0.1% to about 5%, of particulate materials.

### G. Flavonoids

The compositions of the present invention may comprise a flavonoid. The flavonoid can be synthetic materials or obtained as extracts from natural sources, which also further may be derivatized. Examples of classes of suitable flavonoids are disclosed in U.S. Pat. No. 6,235,773, issued to Bissett, and include, but are not limited to, unsubstituted flavanones, methoxy flavanones, unsubstituted chalcones, and mixtures thereof. In one embodiment, the flavonoids are unsubstituted flavanones, unsubstituted chalcone (especially the trans-isomer), their glucosyl derivatives, and mixtures thereof. Other examples of suitable flavonoids include flavanones such as hesperidin compounds (e.g., glucosyl hesperidin), isoflavones such as soy isoflavones, including but not limited to genistein, daidzein, quercetin, and equol, their glucosyl derivatives, 2′,4-dihydroxy chalcone, and mixtures thereof.

The compositions of the present invention may comprise from about 0.01% to about 20%, alternatively from about 0.1% to about 10%, and alternatively from about 0.5% to about 5% of flavonoids.

### H. Hair Growth Regulators

The compositions of the present invention may comprise compounds useful for regulating hair growth. Suitable hair growth regulators include, but are not limited to, hexamidine, butylated hydroxytoluene (BHT), hexanediol, panthenol and pantothenic acid derivates, their isomers, salts and derivatives, and mixtures thereof. The compositions of the present invention may comprise from about 0.0001% to about 20%, alternatively from about 0.001% to about 10%, alternatively from about 0.01% to about 5%, and alternatively from about 0.1% to about 2% of hair growth regulators.

### I. Other Skin Care Actives

The compositions of the present invention further may comprise non-vitamin antioxidants and radical scavengers, minerals, preservatives, phytosterols and/or plant hormones, protease inhibitors, tyrosinase inhibitors, anti-inflammatory agents and N-acyl amino acid compounds.

Suitable non-vitamin antioxidants and radical scavengers include, but are not limited to, BHT (butylated hydroxy toluene), L-ergothioneine (available as THIOTANE™); tetrahydrocurcumin, cetyl pyridinium chloride, carnosine, diethylhexyl syrinylidene malonate (available as OXYNEX™), hexadec-8-ene-1,16-dicarboxylic acid (octadecene dioic acid; ARLATONE™ Dioic DCA from Uniqema), ubiquinone (co-enzyme Q10), tea extracts including green tea extract, yeast extracts (e.g., Pitera®), yeast culture fluid, and combinations thereof.

Suitable minerals include zinc, manganese, magnesium, copper, iron, selenium and other mineral supplements. “Mineral” is understood to include minerals in various oxidation states, mineral complexes, salts, derivatives, and combinations thereof.

Suitable examples of plant sterols (phytosterols) and/or plant hormones include, but are not limited to, sitosterol, stigmasterol, campesterol, brassicasterol, kinetin, zeatin, and mixtures thereof.

Suitable protease inhibitors include, but are not limited to, hexamidine, vanillin acetate, menthyl anthranilate, soybean trypsin inhibitor, Bowman-Birk inhibitor, and mixtures thereof.

Suitable tyrosinase inhibitors include, but are not limited to, sinablanca (mustard seed extract), tetrahydrocurcumin, cetyl pyridinium chloride, and mixtures thereof.

Suitable anti-inflammatory agents include, but are not limited to, glycyrrhizic acid (also known as glycyrrhizin, glycyrrhixinic acid, and glycyrrhetinic acid glycoside), glycyrrhetenic acid, other licorice extracts, and combinations thereof.

Suitable N-acyl amino acid compounds include, but are not limited to, N-acyl phenylalanine, N-acyl tyrosine, their isomers, including their D and L isomers, salts, derivatives, and mixtures thereof. An example of a suitable N-acyl amino acid is N-undecylenoyl-L-phenylalanine is commercially available under the tradename SEPIWHITE® from Seppic (France).

Other useful skin care actives include moisturizing and/or conditioning agents, such as glycerol, petrolatum, caffeine, and urea; yeast extracts (for example, Pitera™); dehydroepiandrosterone (DHEA), its analogs and derivatives; exfoliating agents, including alpha- and beta-hydroxyacids, alpha-keto acids, glycolic acid and octanoyl salicylate; antimicrobial agents; antidandruff agents such as piroctone olamine, 3,4,4′-trichlorocarbanilide (trichlosan), triclocarban and zinc pyrithione; dimethyl aminoethanol (DMAE); creatine; skin lightening agents such as kojic acid, mulberry extract, hydroquinone, arbutin, and deoxy-arbutin; (sunless) tanning agents, such as dihydroxy acetone (DHA); plant-derived materials such as resveratrol; isomers, salts, and derivatives of any of the foregoing; and mixtures thereof.

### Dermatologically Acceptable Carrier

The compositions of the present invention comprise a dermatologically acceptable carrier for the skin care actives materials. The compositions may comprise from about 50% to about 99.99%, alternatively from about 60% to about 99.9%, alternatively from about 70% to about 98%, and alternatively from about 80% to about 95% of the composition.

The carrier can be a wide variety of types, non-limiting examples of which include solutions, dispersions, emulsions and combinations thereof. Herein, “emulsions” generally contain an aqueous phase and an oil phase. The oils may be derived from animals, plants, or petroleum, may be natural or synthetic, and may include silicone oils. Emulsion carriers include, but are not limited to oil-in-water, water-in-oil and water-in-oil-in-water emulsions. In one embodiment, the carrier comprises an oil-in-water emulsion, a water-in-oil emulsion a silicone-in-water emulsion, and/or a water-in-silicone emulsion. The emulsions may comprise from about 0.01% to about 10%, and alternatively from about 0.1% to about 5%, of a nonionic, anionic or cationic emulsifier, and combinations thereof. Suitable emulsifiers are disclosed in, for example, U.S. Pat. No. 3,755,560, U.S. Pat. No. 4,421,769, and McCutcheon's Detergents and Emulsifiers, North American Edition, pages 317-324 (1986).

The carrier further may comprise a conditioning agent and/or a thickening agent. Suitable conditioning agents include a humectant, moisturizer, and/or skin conditioner. The composition may comprise from about 0.01% to about 20%, alternatively from about 0.1% to about 10%, and alternatively from about 0.5% to about 7%, of a conditioning agent. In one embodiment, the conditioning agent is selected from urea, guanidine, sucrose polyester, panthenol, dexpanthenol, allantoin, glycerol, and mixtures thereof.

The compositions of the present invention may comprise from about 0.05% to about 5%, alternatively from about 0.1% to about 4%, and alternatively from about 0.25% to about 3%, of a thickening agent. Suitable classes of thickening agents include, but are not limited to carboxylic acid polymers, crosslinked polyacrylate polymers, polyacrylamide polymers, polysaccharides, gums and mixtures thereof. Non-limiting examples of suitable thickening agents are described in the CTFA International Cosmetic Ingredient Dictionary, 10^{th} Ed. (2004), pp. 2294-96.

The composition further may comprise from about 0.1% to about 20%, alternatively from about 1% to about 10%, and alternatively from about 5% to about 7% of dipropylene glycol monocaprylate, isopropyl lauroyl sarcosinate, and mixtures thereof, suitable for use with oil soluble actives. Dipropylene glycol monocaprylate is commercially available as Caproyl™ 90 (Gattefosse, Gennevilliers, France). Isopropyl lauroyl sarcosinate is commercially available as Eldew™, which can be purchased from Ajinomoto U.S.A., Paramus, N.J. Non-limiting examples of oil-soluble actives suitable for use with dipropylene glycol monocaprylate and/or isopropyl lauroyl sarcosinate include, but are not limited to, tetrahydrocurcumin, tetrahydrocurmin diacetate, glycyrrhizic acid, glycyrrhetinic acid, lauryl p-cresol ketoxime, bis-abolol and ginger extract, alpha-linoleic acid, oil soluble vitamin C, carnosic acid, ursolic acid, proanthocyanidins, green tea polyphenols, oleuropein, xymenynic acid, ethyl p-methoxycinnamate, and lupeol hemisuccinate.

### Optional Components

The compositions of the present invention may contain a variety of other ingredients that are conventionally used in given product types provided that they do not unacceptably alter the benefits of the invention.

The optional components, when incorporated into the composition, should be suitable for use in contact with human keratinous tissue without undue toxicity, incompatibility, instability, allergic response, and the like within the scope of sound judgment. The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, anti-caking agents, antifoaming agents, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, and thickeners.

### II. Methods for Regulating Keratinous Tissue Condition

The present invention describes one or more methods of regulating the condition of mammalian skin and/or of providing a benefit to keratinous tissue. The method may comprise the step of topically applying to mammalian skin in need of regulation a personal care composition of the present invention. In one embodiment, the benefit is selected from the group consisting of reducing the appearance of wrinkles, deep lines, fine lines, crevices, bumps, large pores; increasing the convolution of the dermal-epidermal border; skin lightening; increasing elasticity, decreasing sagging, reducing cellulite; reducing the appearance of under-eye circles, reducing the appearance of discoloration, reducing hyperpigmentation, increasing skin luminosity, and combinations thereof. Alternatively, the benefit is selected from the group consisting of reducing hyperpigmentation, skin lightening, and combinations thereof. In one embodiment, the method comprises the step of selectively applying the composition to an area of skin in need of a benefit, where “selectively” is understood to mean that the composition is applied primarily to the area of skin in need of treatment, for example an age spot, and little or no composition is applied to the surrounding keratinous tissue that does not appear to be in need of treatment.

The composition may be applied by a variety of means, including by rubbing, wiping or dabbing with hands or fingers, or by means of an implement and/or delivery enhancement device. Non-limiting examples of implements include a sponge or sponge-tipped applicator, a swab (for example, a cotton-tipped swab), a pen optionally comprising a foam or sponge applicator, a brush, a wipe, and combinations thereof. Non-limiting examples of delivery enhancement devices include cloths, masks, substrates, gels (including pre-formed gels), temperature change elements, and combinations thereof. The composition may be pre-applied to the applicator, and for example delivered to the user pre-packaged as such, or the user may be instructed to apply the composition to the applicator prior to use. Alternatively, the composition may be stored in an implement, for example, in a cotton-tipped swab. The implement may comprise a separate storage area for the composition, and composition may be transferred to the applicator directly from the storage area, for example, by squeezing and/or breaking or by other suitable means. The composition may be applied to the keratinous tissue by contacting the applicator and composition to the skin. Contact may include, for example, light pressure, dabbing, rubbing, wiping, or any other suitable means. When targeted application is desired, the composition may be applied to the desired area of keratinous tissue by means of a suitable applicator.

The amount of the composition applied, the frequency of application and the period of use will vary widely depending upon the level of components of a given composition and the level of regulation desired. For example, from about 0.01 g composition/cm² to about 1 g composition/cm² of keratinous tissue may be applied. In one embodiment, the compositions are applied at least once daily, where “daily” and “days” mean a 24-hour period. For example, the compositions may be applied daily for 30 consecutive days, alternatively for 14 consecutive days, alternatively for 7 consecutive days and alternatively for 2 consecutive days.

The method may comprise the step of inducing a temperature change in the composition and/or in the keratinous tissue either simultaneously or sequentially with the step of applying the composition. The method further may comprise additional steps which form part of a treatment or application regimen, including the steps of applying at least one additional composition, ingesting one or more dietary supplements, cleansing, etc.

The present invention also contemplates the delivery of energy, via a delivery enhancement device, to keratinous tissue, either simultaneously and/or sequentially (e.g., within 10 minutes) with application of the topical compositions. The energy delivery device may deliver energy in a variety of forms, including but not limited to energy in the form of light, heat, sound (including ultrasonic energy), magnetic energy, electromagnetic energy (including radiofrequency waves and microwaves), mechanical energy (exfoliating or microdermabrasion device), and combinations thereof. In one embodiment, the energy is selected from the group consisting of light energy, heat energy, ultrasonic energy, magnetic energy, electromagnetic energy, mechanical energy, and combinations thereof. The delivery of energy may be continuous, pulsed, modulated, non-modulated, and combinations thereof. In one embodiment, the energy delivery device is hand-held. Alternatively, the energy delivery device is cordless.

The energy may be applied by holding a device within a single area of keratinous tissue, and subsequently moving the device to another area of tissue (or “stamping”). Alternatively, the energy may be applied as the device is continuously moved, or scanned, across the surface of the tissue. The device may be held in substantially continuous contact with the surface of the keratinous tissue, as with laser devices, or may be held at a short distance from the keratinous tissue with the energy directed toward the surface, as with flash lamps.

A temperature change may be simultaneously induced in the keratinous tissue or alternatively, in a compound applied to the surface of the tissue. This temperature change is in addition to any temperature change induced by the delivered energy itself. For example, the keratinous tissue may be slightly warmed prior to delivery of energy, or alternatively, the keratinous tissue may be cooled after delivery of energy.

### III. Kit

The present invention further provides a kit comprising at least one composition described herein. The kit may comprise an outer packaging unit, which in turn may comprise one or more inner packaging units. The inner packaging units may be suitable for a single application, or “unit dose.” The inner and outer packaging units may be of any type suitable for containing, presenting and/or reasonably protecting from damage the contents of the kit. The kit further may comprise an implement, which may be suitable for targeted delivery of the composition to a desired area of keratinous tissue. The composition may be packaged separately from the implement, or may be contained within the implement. The kit further may comprise a plurality of components, including but not limited to, one or more additional compositions, one or more orally ingestible dietary supplements, an implement, a delivery enhancement device, an energy delivery device, a temperature change element, instructions for use of the implement and/or device, instructions for complying with suitable application regimens, instructions for targeted delivery, a substrate, and combinations thereof.

When an implement such as a pen is included, a non-limiting example of which is SHO127 F2 applicator from Shya Hsin™, one example of suitable instructions includes the following. Cleanse skin thoroughly before applying. Apply a thin layer of the product on the most visible dark spots, twice daily. To use: Turn the mechanism at the base of the pen until a drop of product appears at the end of the tip. Apply the concentrate on the most visible facial age spots, lightly massage product in by using the tip of the applicator to help penetration. If desired, you may follow ip with your moisturizer or foundation makeup. Sun exposure should be limited by using a sunscreen agent, a sun blocking agent, or protective clothing.

When an implement such as a swab is included, a non-limiting example of which is an XPress Tip™ from SwabPlus™, one example of suitable instructions includes the following. Directions: Cleanse skin thoroughly before applying. Apply a thin layer of the product on the most visible dark spots, twice daily. To use: Hold swab with soft tip pointing up, slowly push the upper tube down into the lower tube. Treatment product will flow up into the soft tip, continue to push product until desired amount of product is visible at the swab tip. Apply the concentrate on the most visible facial age spots, lightly massage product in by using the tip of the applicator to help penetration. If desired, you may follow up with your moisturizer or foundation makeup. Sun exposure should be limited by using a sunscreen agent, a sun blocking agent, or protective clothing. Dispose of swab after use.

### IV. Article of Commerce

The present invention also provides articles of commerce comprising at least one personal care composition described herein, and advertisement material pertaining to the personal care composition comprises indicia and/or an image which communicates to a consumer that the personal care composition can be used in conjunction with an implement and/or energy delivery device for regulating the condition of and/or providing a benefit to mammalian keratinous tissue. The advertisement material may comprise images comparing the appearance of a person prior to use of the composition to the appearance of the same person after use of the composition. In one embodiment, the benefit is selected from the group consisting of skin lightening, reducing hyperpigmentation, and combinations thereof.

### V. Method of Marketing

The present invention also provides methods of marketing personal care compositions for preventing, retarding, and/or treating uneven skin tone. One preferred method of marketing such compositions includes making available to a consumer a personal care composition described herein and communicating to the consumer that the topical application of the personal care composition may improve the consumer's skin color. The manner in which the communication is conveyed to the consumer is non-limiting. By way of example only, the communication can be effected by known advertisement techniques, such as, television, internet and magazine advertisements. The communication may be a point-of-sale technique, such as, for example, a shelf and/or floor affixed communication. Additionally or alternatively, he communication may take the form of indicia (text, symbols, colors, shades, figures, and the like) disposed in and/or on packaging of the personal care compositions.

Methods of conducting business are also contemplated by the present invention. One preferred method includes the step of communicating to a consumer a comparison between a first personal care composition comprising an active discussed herein and a second personal care composition that does not include the noted active. The comparison may relate to skin tone, skin lightening, skin whitening, pigmentation, among other parameters associated with regulating mammalian keratinous tissue conditions.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

Whereas particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a) at least one skin care active selected from the group consisting of acetyl triethyl citrate, gluconolactone, arginine glutamate and salts, isomers, derivatives, and mixtures of any of the foregoing; and
b) a dermatologically acceptable carrier; and
c) at least one additional skin care active selected from the group consisting of vitamin B compounds, vitamin E compounds, vitamin C compounds, vitamin D compounds, peptides, sugar amines, protease inhibitors, sunscreens, desquamation agents, chelators, skin lightening compounds, non-vitamin antioxidant radical scavengers, phytosterols, hesperidin compounds, retinoid compounds, plant hormones, protease inhibitors, tyrosinase inhibitors, anti-inflammatory agents, topical anesthetics, anti-cellulite agents, sunless tanning agents, N-acyl amino acid compounds, yeast extract, and salts, isomers, derivatives and mixtures of any of the foregoing.

2. The personal care composition of claim 1, wherein the composition comprises from about 0.01% to about 10% of the skin care active.

3. The composition of claim 1, wherein the additional skin care active is selected from the group consisting of niacinamide, tocopheryl nicotinate, vitamin E acetate, N-acetyl-D-glucosamine, glucosyl hesperidin, hexamidine compounds, ascorbyl glucoside, palmitoyl-lysine-threonine-threonine-lysine-serine, butylated hydroxytoluene, cetyl betaine, bisabolol, green tea extract, theophylline, undecenoyl phenylalanine, cetyl pyridinium chloride, dihydroxyacetone, panthenol, butyl methoxydibenzoylmethane, glycyrrhizic acid, glycyrrhetinic acid, butylated hydroxyanisole, tetrahydrocurcumin, ergothioneine, octadecenedioic acid, retinyl propionate, and salts, isomers, derivatives, mixtures of any of the foregoing.

4. The personal care composition of claim 1, wherein the carrier comprises from about 0.1% to about 20% of dipropylene glycol monocaprylate, isopropyl lauroyl sarcosinate, and mixtures thereof.

5. A method of providing a benefit to mammalian keratinous tissue, comprising the step of applying to the keratinous tissue a personal care composition comprising:
a) at least one skin care active selected from the group consisting of acetyl tributyl citrate, gluconolactone, arginine glutamate, and salts, isomers, derivatives, and mixtures of any of the foregoing;
b) a dermatologically acceptable carrier, and
c) at least one additional ingredient selected from the group consisting of vitamin B compounds, vitamin E compounds, vitamin C compounds, vitamin D compounds, peptides, sugar amines, protease inhibitors, sunscreens, desquamation agents, chelators, skin lightening compounds, non-vitamin antioxidant radical scavengers, phytosterols, hesperidin compounds, retinoid compounds, plant hormones, protease inhibitors, tyrosinase inhibitors, anti-inflammatory agents, topical anesthetics, anti-cellulite agents, sunless tanning agents, N-acyl amino acid compounds, yeast extract, and salts, isomers, derivatives and mixtures of any of the foregoing; and a dermatologically acceptable carrier

6. The method according to claim 5, wherein the composition comprises at least one additional ingredient selected from the group consisting of niacinamide, tocopheryl nicotinate, vitamin E acetate, N-acetyl-D-glucosamine, glucosyl hesperidin, hexamidine compounds, ascorbyl glucoside, palmitoyl-lysine-threonine-threonine-lysine-serine, butylated hydroxytoluene, cetyl betaine, bisabolol, green tea extract, theophylline, undecenoyl phenylalanine, cetyl pyridinium chloride, dihydroxyacetone, panthenol, butyl methoxydibenzoylmethane, glycyrrhizic acid, glycyrrhetinic acid, butylated hydroxyanisole, tetrahydrocurcumin, ergothioneine, octadecenedioic acid, retinyl propionate, and salts, isomers, derivatives and mixtures of any of the foregoing.

7. The method of claim 5, wherein the benefit is selected from the group consisting of reducing the appearance of wrinkles, deep lines, fine lines, crevices, bumps, large pores; increasing the convolution of the dermal-epidermal border; skin lightening; increasing elasticity, decreasing sagging, reducing cellulite; reducing the appearance of under-eye circles, reducing the appearance of discoloration, reducing hyperpigmentation, increasing skin luminosity, and combinations thereof.

8. The method of claim 9, wherein the benefit is skin lightening.

9. The method of claim 9, wherein the benefit is reducing hyperpigmentation.

10. The method of claim 6, wherein the composition is selectively applied to an area of skin in need of the benefit.

11. The method of claim 10, wherein the composition is selectively applied to a hyperpigmented area of skin.

12. The method of claim 12, wherein the composition is applied with an implement selected from the group consisting of a swab, a pen, and combinations thereof.

13. The method of claim 5, further comprising at least one additional step selected from the group consisting of delivering energy to the keratinous tissue by means of an energy delivery device, applying at least one additional composition, ingesting one or more dietary supplements, cleansing, and combinations thereof.

14. The method of claim 14, wherein the energy is selected from the group consisting of light energy, heat energy, ultrasonic energy, magnetic energy, electromagnetic energy, mechanical energy, and combinations thereof.

15. A kit comprising:
a) at least one skin care active selected from the group consisting of acetyl triethyl citrate, gluconolactone, arginine glutamate, and salts, isomers, derivatives, and mixtures of any of the foregoing; and a dermatologically acceptable carrier;
b) at least one additional component select from the group consisting of at least one additional composition, at least one orally ingestible dietary supplement, an implement, a delivery enhancement device, a temperature change element, instructions for complying with suitable application regimens, and combinations thereof; and;
c) instructions for complying with a regimen to provide a benefit to keratinous tissue.

16. A kit according to claim 15, wherein the instructions indicate to selectively apply the composition to an area of keratinous tissue in need of a benefit.

17. An article of commerce, comprising:
a) a personal care composition comprising the personal care composition of claim 1;
b) at least one packaging for the personal care composition; and
c) advertisement material pertaining to the personal care composition comprising indicia which communicates that the personal care composition can be used in conjunction with an energy delivery device for providing a benefit to mammalian keratinous tissue.

18. The article of commerce of claim 17, wherein the benefit is selected from the group consisting of skin lightening, reducing hyperpigmentation, and combinations thereof.
